(19) United States
(12) Patent Application Publication
(10) Pub. No.: US 2025/0017855 A1
(43) Pub. Date: Jan. 16, 2025

(54) Pharmaceutical Preparation for Topical Delivery of Botulinum Toxin

(71) Applicant: Institute of Advanced Sciences, North Dartmouth, MA (US)

(72) Inventors: Bal Ram Singh, Rehoboth, MA (US); Raj Kumar, Dartmouth, MA (US)

(73) Assignee: Institute of Advanced Sciences, North Dartmouth, MA (US)

(21) Appl. No.: 18/352,248

(22) Filed: Jul. 14, 2023

(51) Int. Cl. A61K 9/107; A61K 9/00; A61K 38/48; A61K 45/06

(52) U.S. Cl. CPC A61K 9/1075; A61K 9/0014; A61K 38/4893; A61K 45/06

(57) ABSTRACT

A pharmaceutical composition, method of making and treating, comprising one or more proteins of Clostridium botulinum in a nano-emulsion for topical delivery to treat primarily skin disorders. In an embodiment, the nano-emuslisons are encapsulated microspheres and nanospheres comprising: propylene glycol, phenoxyethanol, sodium hyaluronate, caprylic/capric triglyceride, hydrogenated castor oil, span-80, and saponin, with water. A method of making the nanoemulsions, comprises: (1) preparing a solution A by mixing sodium hyaluronate, propylene glycol, Tween-80 and saponin in water by continuous stirring; (2) preparing a solution B by mixing phenoxy ethanol and carylic acid, or capric triglyceride, together; (3) mixing B into A by continuous stirring for 15 minutes to form solution C; (4) mixing Botulinum toxin and 10 mM Sodium Phosphate buffer to a pH of 7.1, and adding to solution C; and (5) stirring solution C at room temperature for 15-20 minutes.

### BACKGROUND OF THE INVENTION

There are 7 major serotypes of Botulinum toxins (BoNTs), each consisting of a primarily two major domains: binding domain (Heavy chain (HC)) and a catalytic domain (Light chain (LC)), linked through a disulfide bond (Montecucco and Schiavo 1995). Upon binding specifically to the presynaptic nerve membrane, BoNT is internalized through endocytosis, and the LC is translocated through a membrane pore formed by the translocation domain (TD) of the HC (Li and Singh 2000). The LC of each serotype of BoNT is a zinc-metalloprotease and acts as an endopeptidase with remarkable substrate specificity requiring a substantially long peptide sequence, depending on the serotype (Segelke et al. 2004). This is unique to BoNTs as other microbial metalloproteases can recognize sequences as short as a dipeptide (Segelke et al., 2004; Silvaggi et al. 2007). Serotype A cleaves SNAP-25 (25-kDa synaptosome associated protein), serotypes B, C, D, F, and G cleave synaptobrevin, and serotype C also cleaves syntaxin, all these substrates involved in the exocytosis process (Li and Singh 1999; Kukreja and Singh 2007).

BoNTs are secreted from the Clostridium botulinum bacteria in the form of multimeric complexes, with a set of non-toxic proteins coded for by genes adjacent to the neurotoxin gene (Inoue et al., 1996; Singh et al., 2014). The geneus Clostridium has more than one hundred and twenty seven species. Their grouping is based on the morphology and function. Botulinum complex size ranges from 300 kDa to 900 kDa and exist in three progenitor toxin forms or variants: M (medium), L (large) and LL (extra-large) forms. The M form consists of neurotoxin (150 kDa) and a nontoxic protein component (120 kDa), which is called neurotoxin binding protein (NBP) (Singh et al., 1995) or nontoxic non-hemagglutinin component (NTNH) (East and Collins, 1994) with 12S molecular size (the molecular size of complex forms is expressed as sedimentation equilibrium values). Common to all serotypes the L form has molecular weight of about 500 kDa and a molecular size of 16S. The LL form is about 900 kDa and 19S for type A. It is notable that each of the serotypes of the botulinum toxin consists of 150 kDa toxic protein, surrounded by a group of neurotoxin associated proteins (NAPs), providing them a common form of isolated toxin or a complex of the toxin and NAPs. Other variants of toxin derived biomolecules could be heavy chain conjugated with therapeutic drugs or biomolecules, engineered light chain or toxin or any combination thereof.

Currently, major approved BoNT therapeutic products include BoNT/A complex (marketed as Botox® and Dysport®), BoNT/B complex (marketed as Myobloc® and Neurobloc®), and isolated BoNT/A without NAPs (marketed as Xeomin®). Although there is no therapeutic role defined for NAPs, these may play a role in the stability of the BoNT formulation and in diffusion of the injected BoNT for therapeutic purposes (Carli et al., 2009; Shone et al., 2011). In BoNT/A complex preparations, either sodium chloride (Botox®, Allergan, Inc.) or lactose (Dysport®, Ipsen, Ltd.) is added to protect the steric conformation of BoNT (Panicker and Muthane, 2003). Human serum albumin is also added to prevent loss from surface adsorption. The toxin is then dried either with freezing (Dysport®) or without freezing (Botox®, Allergan, Inc.) (Panicker and Muthane, 2003). These, as well as the pure BoNT/A product Xeomin®, are lyophilized products that are reconstituted with saline solution and maintained near physiological pH.

Botulinum toxin A complex is the most lethal molecule known to human with LD50 in mice (1 unit), and is ranging from 25-50 picograms. In molar terms, Botulinum toxin A complex is 1.8 billion times more lethal than diptheria, about 600 million times more lethal than sodium cyanide, about 30 million times more lethal than than cobra toxin, and about 12 million times more lethal than cholera. Symptoms of botulinum toxin intoxication includes: vomiting, fever, and difficulty breathing, which progresses to difficult walking, swallowing, and speaking, and to impaired function of diaphragm muscles, and finally death. There are four steps of intoxication of botulinum toxin: i) the toxin binds to the presynaptic membrane of the target neuron; ii) internalization of toxin by receptor mediated endocytosis; iii) tranlocation of toxin to the cytosol of presynapticaxons; and iv) cleavage of SNARE (Soluble N-ethylmaleimide-sensitive factor activating protein receptor) proteins to inhibit acetylcholine release. Botulinum toxin also inhibits potassium cation induced release of both acetylcholine and norepinephrine from primary cell cultures. Not only acetylcholine release is inhibited by botulinum toxin, it also impaired the release of other neurotranmitters, such as glycine, glutamate, CGRP, substance P and GABA (Hanermann et al., 1988; Pearce et al., 1993; Sanchez et al,, 1997; Bigalke et al., 1985, 1986; Jankovic, 1994).

### Administration of Botulinum Toxin

In spite of its toxic effects, Botulinum toxin is considered as a wonder drug. The FDA (Food and Drug Administration) approved botulinum toxin for chronic migraine, cervical dystonia, blepharospasm, strabismus, hyperhidrosis, cosmetics, urinary incontinence from detrusor overactivity, and hemifacial spasms. Other than approved indications, botulinum toxin is frequently used as a off-label use for more than 800 neuro-muscular indications. Botulinum toxin is frequently administered to the patients for treatment by injecting a composition containing botulinum toxin into affected muscles and glands. Generally, the effective dose depends on the mass of muscle being injected. In general, the larger the muscle, then the higher will be the dose; however, the susceptibility to the toxin varies. Local injection of toxin will weaken overactive muscles and control hypersecretion glands supplied by cholinergic neurons. Local injections have been shown to be effective in conditions like achalasia, chronic anal fissure and hyperhidrosis. Other modes of administration have been considered, such as topical administration. Although the major focus for topical administration has been to deliver small molecules, delivering large molecules, such as proteins, could be challenging. In addition, delivery of large molecules frequently require other mediative techniques, such as iontophoresis or ultrasound or microneedle. None of the formulation exists that deliver the botulinum toxin without the help of any mediative techniques. What is needed therefore is a formulation or composition containing a therapeutically effective amount of botulinum toxin that will permeate the botulinum toxin through the skin and demonstrate its efficacy in exerting the desired therapeutic effect.

### Skin Permeability of Protein Molecules

Skin is the largest organ of the body, protects humans from microbes, external pathogens, and other external environmental threats. The other role of skin includes regulation of body temperature, stores water and fat, prevents water loss, involved in sensory and motor responses through sensations of touch, heat and cold, and involved in the production of vitamin D. Human skin is made of 70% water, 27.5% protein, 2% fat, 0.5% mineral salts and trace elements.

Skin has three layers: a) the epidermis (the outermost layer of skin); b) the dermis (beneath the epidermis); and c) the hypodermis (the deeper subcutaneous dermis). The epidermis is the top layer of skin and the thinnest layer of the epidermis, and it contains three types of cells: squamous cells (stratum corneum), basel cells (second layer of epidermis), and melanocytes (base of epidermis). The dermis is the middle layer of the skin containing blood vessels, lymph vessels, hair follicles, sweat glands, collagen bundles, fibroblasts, nerves, pain and touch receptors, and sebaceous glands. The dermis provides flexibility and strength to the skin and is held together by a protein called collagen. The hypodermis layer is the deepest layer of skin made of collagen and fat cells and acts as a shock absorber.

The mean thickness of epidermis was found in the range of 76.9±26.2 to 267.4±120.6 μm. Dermis is the thickest part of the skin. The mean dermal thickness ranged from 2115±946.4 to 5888±2422.2 μm (Oltulu et al., 2018).

One of the important functions of skin is to prevent the water loss and provide the barrier for the transport of the molecules. The body would rapidly dehydrate in the absence of skin. Although most substances cannot penetrate the skin, several strategies have been developed to increase the permeability of small molecules and small peptides.

Several formulations and techniques are available to increase the permeabiity of molecules through the skin. However, permeability of big molecule, such as proteins, through skin is difficult. The present invention provides a method for efficient delivery of botulinum toxin through skin as a topical medicine. In addition, the present invention includes a method for formulating the toxin for topical applications.

### SUMMARY OF THE INVENTION

Various embodiments of the present invention comprise a pharmaceutical composition, method of making and treating, comprising one or more proteins of Clostridium botulinum in a nano-emulsion for topical delivery to treat primarily skin disorders. In an embodiment, the nano-emuslisons are encapsulated microspheres and nanospheres comprising: propylene glycol, phenoxyethanol, sodium hyaluronate, caprylic/capric triglyceride, hydrogenated castor oil, span-80, and saponin, with water. A method of making the nanoemulsions, comprises: (1) preparing a solution A by mixing sodium hyaluronate, propylene glycol, Tween-80 and saponin in water by continuous stirring; (2) preparing a solution B by mixing phenoxy ethanol and caprylic acid, or capric triglyceride, together; (3) mixing B into A by continuous stirring for 15 minutes to form solution C; (4) mixing Botulinum toxin and 10 mM Sodium Phosphate buffer to a pH of 7.1, and adding to solution C; and (5) stirring solution C at room temperature for 15-20 minutes.

The main aspect of present invention is to provide a pharmaceutical composition to formulate botulinum toxin for topical applications.

Another aspect of the present invention is the use of propylene glycol using the w/v ratio between 0.5 to 5%.

Another aspect of the present invention is the use of phenoxyethanol using the w/v ratio between 0.1 to 1%.

Another aspect of the present invention is the use of saponins using the ratio between w/v 1 to 10%.

Another aspect of the present invention is the use of sodium hyaluronate using the w/v ratio between 0.1 to 1%.

Another aspect of the present invention is the use of caprylic/capric triglyceride using the w/v ratio between 0.5 to 5%.

Another aspect of the present invention is the use of hydeogenated castor oil 40 using the w/v ratio between 0 to 10%.

Another aspect of the present invention is the use of Tween-80 using the w/v ratio between 0.1 to 1%.

Another aspects of the present invention is the use of water.

Another aspects of the present invention is the percentage of water is between 80 to 95%.

Another aspect of the present invention is the formation of nanoemulsions by mixing the above components.

Another aspect of the present invention is to mix any of the serotypes of the botulinum toxin (e.g., type A) or botulinum toxin complex (e.g., type A) with the above nano-emulsion.

Another aspect of the present invention is the method of encapsulation of toxin or complex comprising the steps of:
a. Formation of nano-emulsion by mixing water soluble components (solution A) and oil soluble components (solution B);
b. Botulinum toxin A (or other serotypes)+10 mM Sodium Phosphate buffer, pH=7.1 (solution C)
c. Mix solutions obtained from step a and step b.
d. After mixing, stirring the solution for 15-20 min at room temperature (solution D).

More specifically to the method of preparing: the method of preparing an encapsulation of a toxin or complex, comprising the steps:
a. preparing a solution A by mixing sodium hyaluronate, propylene glycol, Tween-80 and saponin in water by continuos stirring;
b. preparing a solution B by mixing phenoxy ethanol and caprylic acid or capric triglyceride, together;
c. mixing solution B into solution A by continuous stirring for 15 minutes to form solution C;
d. mixing appropriate amount of Botulinum toxin in 10 mM Sodium Phosphate buffer to a pH of 7.1, and adding to solution C; and
e. stirring the final solution obtained in step d at room temperature for 15-20 minutes.

Another aspect of the present invention is the formulation preparation in the form of emulsion or suspension.

Another aspect of the present invention is the use of preservatives in emulsions.

Another aspect of the present invention is the use of a lubricant, such as sodium hyaluronate.

Another aspect of the present invention is the pharmaceutical composition comprises one or more excipients, such as oil solution, mixed glycerides, water-soluble co-solvents and surfactant (such as hydrogenated castor oils).

Another aspect of the present invention is the pharmaceutical composition comprises one or more excipients, such as humectants (glycerin, lecithin or propylene glycol) and emolliants (zinc oxide, white petrolatum, dimethicone, lanolin etc.).

Another aspect of the present invention is the pharmaceutical composition further comprises of one or more proteins (e.g. Clostridium botulinum) within the pharmaceutical composition are emulsified and mixed with one or more of: retinoids, alpha hydroxyl acids, hyaluronic acid and/or its sodium salt, resveratrol, stem cells, EGFs (epidermal growth factors), KGFs (keratinocyte growth factors), FGFs (fibroblast growth factors), HGH (human growth hormones), niacinamide, aloe vera, allantoin and therapeutic agents.

Another aspect of the present invention, the pharmaceutical composition further comprises surface active agents, chelating agents, salicylates, anti-inflammatory agents, antibacterial agents, antifungal agents or phenothiazines.

Another aspect of the invention, the pharmaceutical composition further comprises of other associated proteins of botulinum toxin complex, such as NBP, HA proteins or P-80, individually or any combination thereof.

Another aspect of the present invention is the therapeutic efficacy of the formulated botulinum toxin or botulinum toxin complex.

Another aspect of the present invention is the pharmaceutical composition is a lyophilized or gel or cream or semi-liquid form.

Another aspect of the present invention is the pharmaceutical composition is stabilized at a pH in between 5.5 and 8.0.

Another aspect of the present invention is the pharmaceutical composition is used for topical application to treat a variety of diseases, disorders, and/or to act as an analgesic.

### BRIEF DESCRIPTION OF THE DRAWINGS

FIG. 1: The BoNT gene and its associated protein together form a polycistronic transcription unit. The diagram represents the locus of BoNT gene, NBP, the hemaggutinin gene and the loci of NAPs, and also the locus of transcription regulator for BoNT gene cluster. There are two types of gene cluster. Type I lacks for Orf− (negative) but contains hemagglutinin (HA+). Type II is complementary form of type I (Kumar et al., 2015).

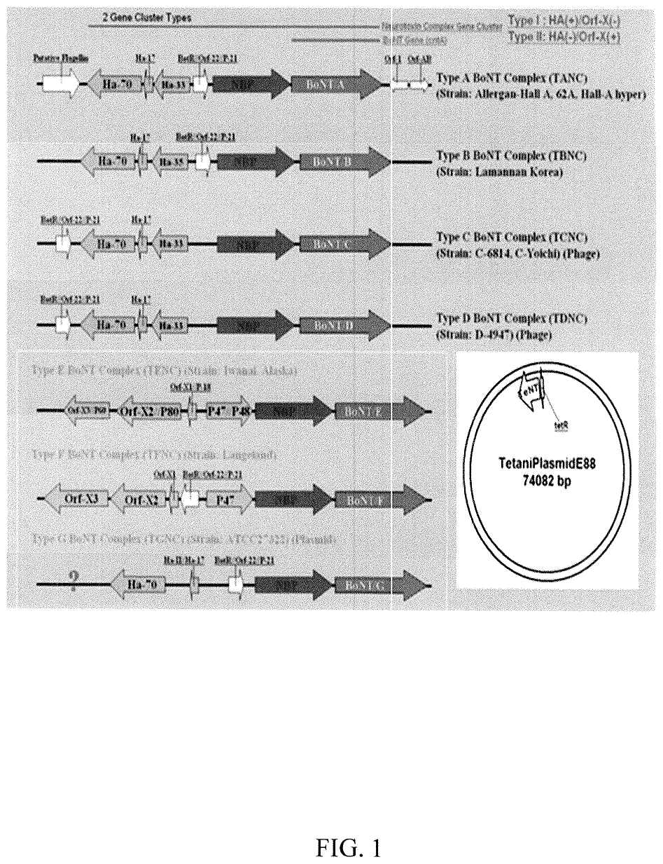

FIG. 2: DAS score of BoNT/A topical formulations. 25, 50 and 100 units of BoNT/A applied topically to the gastrocnemius muscles of the hind leg of each mice. Control was untreated mice and vehicle control was only formulating agent applied topically to the gastrocnemius muscles of the hind leg of each mice. Standard deviation were taken for 5 mice for each experiment.

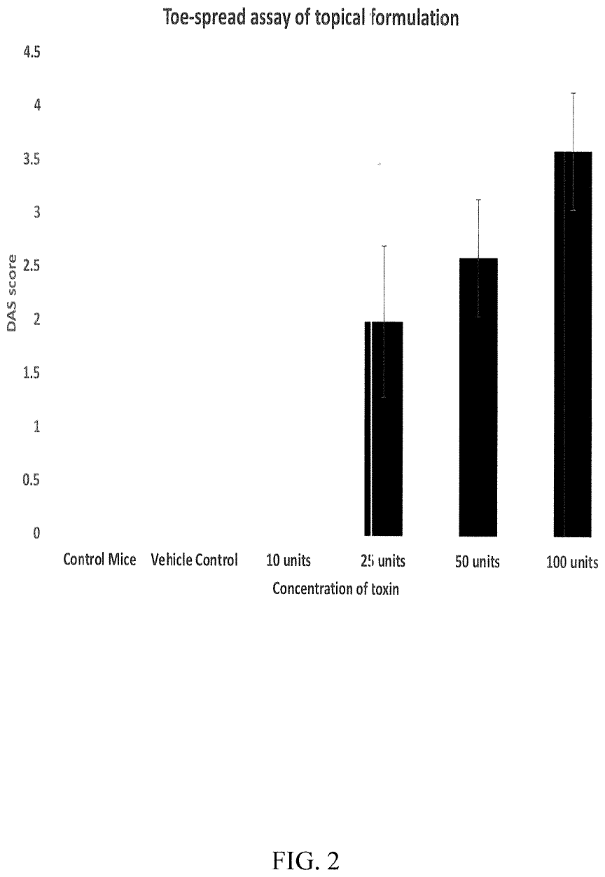

FIG. 3: Recovery assay of topical formulations (Toe-spread assay). All the mice were applied topically with different units of BoNT/A toxin (25, 50 and 100 units). Formulations were applied to the gastrocnemius muscles of the hind leg of each mice and score were recorded by three independent observers for 10 days (every 24 hr interval).

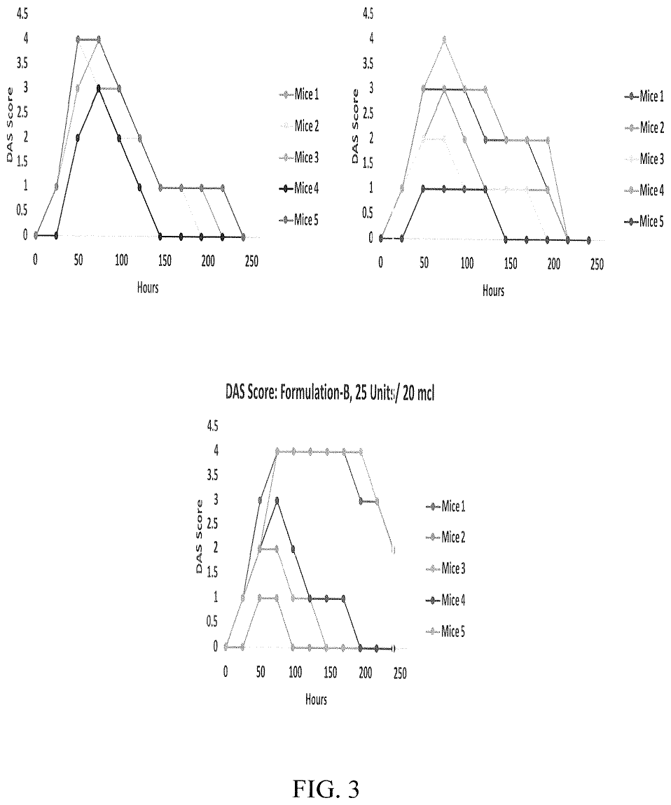

FIG. 4: Recovery assay of topical formulations (Rotarod assay). All the mice were applied topically with different units of BoNT/A toxin (25, 50 and 100 units). Formulations were applied to the gastrocnemius muscles of the hind leg of each mice and score were recorded by three independent observers for 10 days (every 24 hr interval).

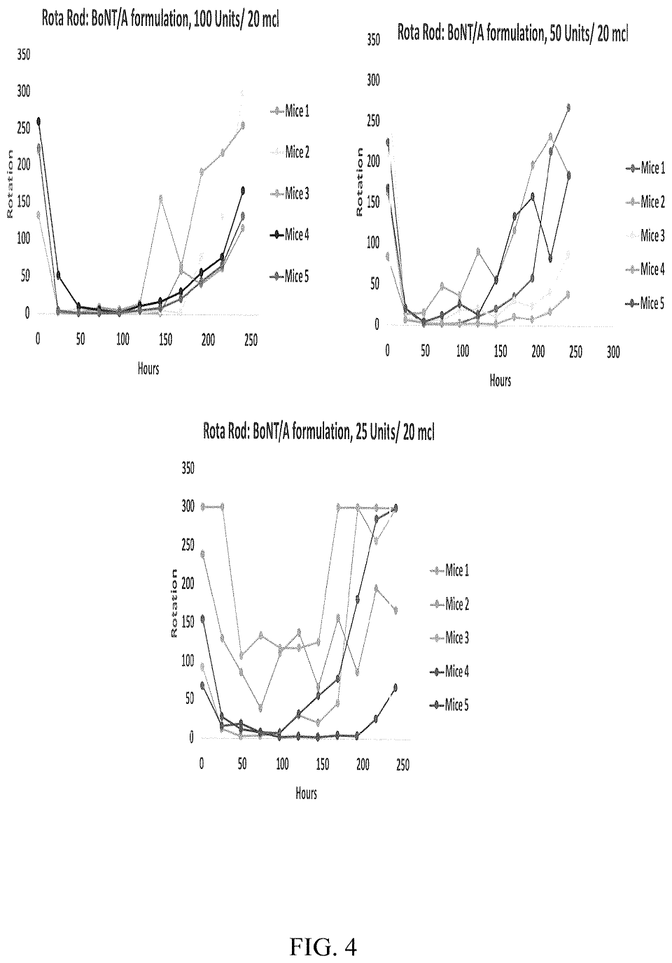

FIG. 5: DAS-score of toe-spread assay. Representative figure of each DAS score from the experiment, including control, after 48 hr of the topical formulation application on the gastrocnemius muscles.

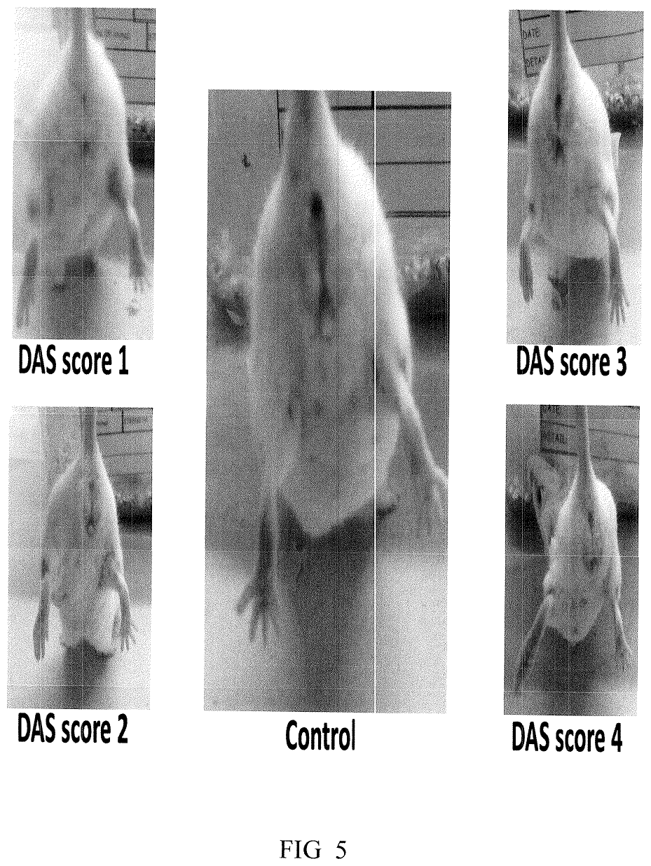

### DETAILED DESCRIPTION OF THE INVENTION

Described herein are formulations, pharmaceutical formulations, and methods of preparation. These pharmaceutical formulations may be prepared by the processes described herein. In some variations the therapeutic agent is botulinum toxin and botulinum toxin complex (e.g., for example from type A Clostridium botulinum).

In some variations the pharmaceutical formulations described herein can be used for the treatment, prevention, inhibition, delaying onset of, or causing regression of one or more neuro-muscular diseases and conditions or treatment of wrinkle or used in cosmetic treatments. In some variations the diseases or conditions include neuronal regeneration/sprouting, disease involving muscle movement, various wounds, scars and gastrointestinal symptoms.

Botulinum neurotoxin is a large protein toxin (approximately 150 kDa) that is able to bind and internalize to motor neurons very specifically. BoNTs are produced by Clostridium botulinum along with several neurotoxins associated polypeptides (proteins) (NAPs). The toxin with NAPs is referred herein as a “complex toxin”. There are three forms of toxin; LL, L and M complex. Other variants of toxin derived biomolecules could be heavy chain conjugated with threpeutic drugs or biomolecules, engineered light chain or toxin or any combination thereof.

The present invention provides the method to formulate toxin for topical administration. The method of preparing he nano-emulsions for topical use comprises the following general steps: 1) mixing of components for the preparation of nano-emulsion; 2) mixing of, for example, BoNT/A or BoNT/A complex with nano-emulsion; 3) permeability of the formulation including BoNT/A in the mice model to demonstrate the desired effect when applied topically; and 4) recovery experiment demonstrating the efficacy of the formulation and recovery of affected muscles after topical administration of the formulation comprising BoNT/A toxin.

In the first part of the present application, a nano-emulsion formulation of pure botulinum toxin A (150 kDa; BoNT/A) was made by mixing pure BoNT/A toxin in the nano-emulsion, as an example. Nano emulsion was prepared by: a) mixing sodium hyaluronate, propylene glycol, Tween-80 and saponin in water by continuos stirring (solution A); b) either mixed phenoxy ethanol, and carylic acid/capric triglyceride together (without hydrogenated castor oil) or in hydrogenated castor oil (solution B); and c) mixed solution B into solution A by continuous stirring for 15 min (solution C). Prepared the desired concentration of pure BoNT/A in 10 mM Sodium Phosphate buffer, pH=7.1. Added BoNT/A solution in solution C and stirred the solution at room temperature for 15-20 min (solution D).

In the second part, formulation is applied topically on gastrocnemius muscle of mice. Three concentrations were used; 25, 50 and 100 units (1 unit=1 LD₅₀=25 μg of Balb c mice) of pure BoNT/A in the above formulation (solution D). For this, Balb C mice were used and housed 5 mice per cage, under 12:12-h light:dark cycle in paper chip bedding. Water and rodent chow were used for food supplement. Room temperature and humidity were maintained between 22-26° C. and 50-70%, respectively. The toe-spread reflex was observed by lifting the mouse by the base of the tail, with free hind limb, and allowing the forelimbs to rest on the workbench. Toe spread was observed for 24 hr to 48 hr. Toe-spread data were analyzed using DAS (digital abduction score) score. Zero DAS score indicates complete spread of toe and DAS score four indicated complete paralysis. Formulations were applied on left hind limb of the mice and vehicle control (solution C as a control) was applied to the right hind limb of the mice.

In the third part of the present invention, formulation is applied on gastrocnemius muscle of mice. Three concentrations were used; 25, 50 and 100 units (1 unit=1 LD₅₀=25 μg/kg of Balb c mice) of pure BoNT/A in the above formulation (solution C). For this Balb C mice were used and housed 5 mice per cage, under 12:12-h light:dark cycle in paper chip bedding. Water and rodent chow were used for food supplement. Room temperature and humidity were maintained between 22-26° C. and 50-70%, respectively. The toe-spread reflex was observed by lifting the mouse by the base of the tail, with free hind limb, and allowing the forelimbs to rest on the workbench. Efficacy of the formulation for skin peremeation were analyzed by two assay; toe-spread assay and rota-rod assay. Toe spread and rotarod assay were observed for every 24 hr for 10 days. Formulations were applied on left hind limb of the mice and vehicle control (solution C as a control) was applied to the right hind limb of the mice. DAS (digital abduction score) score as follows: Zero DAS score indicates complete spread of toe and DAS score four indicated complete paralysis. BoNT/A formulations were in solution C was applied in both the hind limbs and monitored for 10 days. DAS score was measured as before. For rota rod experiment, each group of trained mice underwent on rotarod and number of rotation mice were able to remain on the rotarod were recorded. Mice were analyzed every day for 10 days for DAS score and number of rotation.

### Pharmaceutical Composition Components

The various embodiments of the pharmaceutical composition comprises: one or more proteins of Clostridium botulinum in a nano-emulsion for topical delivery. The one or more proteins comprises one or more from botulinum toxin A (BoNT/A), BoNT/B, BoNT/C, BoNT/D, BoNT/E.BoNT/F, or BoNT/G, or their variants, its complex, or its other forms, or other proteins of Clostridium botulinum.

In an embodiment, the pharmaceutical composition is stabilized at a pH in between 5.5 and 8.0.

In an embodiment, the pharmaceutical composition comprises nano-emuslisons with encapsulated microspheres and nanospheres containing propylene glycol (0.5 to 5% w/v), phenoxyethanol (0.1 to 1% w/v), sodium hyaluronate (0.1 to 1% w/v), caprylic/capric triglyceride (0.5 to 5% w/v), hydrogenated castor oil (0 to 10

% w/v), span-80 (0.1 to 1% w/v) and saponin (1 to 10% w/v) with water.

In an embodiment, the pharmaceutical composition further comprises other therapeutic proteins including biomolecules, vaccines, antibodies and protein hormones.

In an embodiment, the pharmceutical composition further comprises surface active agents, chelating agents, salicylates, anti-inflammatory agenets, antibacterial agents, antifungal agents, antiviral agents or phenothiazines and bioactive peptides (e.g. pentapeptide KTTKS, tetrapeptide GQPR, hexapeptide argireline, tripeptide GHK, Snap-8 octapeptide and oligopeptides).

In an embodiment, the pharmaceutical composition further comprises one or more oils, e.g.: oil triglyceride, ethyl icosapentate, castor oil, tocopherol nicotinate, teprenone, indomethacin franesil, soy-bean oil, tea oil, sunflower seed oil, vegetable oil, fish oil, sesame oil, soybean oil, tea oil, sunflower seed oil, vegetable oil, fish oil, sesame oil, soy-bean oil, tea oil, sunflower seed oil, vegetable oil, fish oil, sesame oil, soy-bean oil, tea oil, sunflower seed oil, vegetable oil, fish oil, sesame oil, Labrafac Lipophile WL 1349 oil, and dronabinol).

In an embodiment, the pharmaceutical composition further comprises one or more excipients, e.g.: oil solution, mixed glycerides, water-soluble co-solvents and surfactants (Permulen TR1, Permulen TR2, RH-40, Tween-80, and Tween-60), and self-made creams.

In an embodiment, the pharmaceutical composition further comprises humectants (e.g. propylene glycol or lecithin), and emolliants (zinc oxide or dimethicone).

In an embodiment, the pharmaceutical composition further comprises a stabilizer, e.g. serum albumin or IgG.

In an embodiment, the pharmaceutical composition is formulated as a lyophilized powder, lotion, serum or gel form.

In an embodiment, the one or more proteins (e.g. Clostridium botulinum) within the pharmaceutical composition are emulsified and mixed with one or more of: retinoids, alpha hydroxyl acids, hyaluronic acid and/or its sodium salt, resveratrol, stem cells, EGFs (epidermal growth factors), KGFs (keratinocyte growth factors), FGFs (fibroblast growth factors), HGH (human growth hormones), niacinamide, aloe vera, allantoin and therapeutic agents.

Method of Use: the pharmaceutical composition is able to effectively treat local and systemic conditions, e.g.: neuromuscular, gastrointestinal, diabetic, cardiovascular, reproductive issues, skin problems; and to work as a local aesthetic; wherein it is administered orally, injected intramuscularly, or topically (preferred).

### CONCLUSION

It will be appreciated that the methods and compositions of the present disclosure can be incorporated in the form of a variety of embodiments, only a few of which are disclosed herein. It will also be apparent for the expert skilled in the field that other embodiments exist and do not depart from the spirit of the invention. Thus, the described embodiments are illustrative and should not be construed as restrictive.

Accordingly, the preceding exemplifications merely illustrate the principles of the various embodiments. It will be appreciated that those skilled in the art will be able to devise various arrangements which, although not explicitly described or shown herein, embody the principles of the embodiments and are included within its spirit and scope. Furthermore, all examples and conditional language recited herein are principally intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the various embodiments, therefore, is not intended to be limited to the exemplary embodiments shown and described herein.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Or, the technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed.

The term “a” or “an” can refer to one of or a plurality of the elements it modifies (e.g., “a reagent” can mean one or more reagents) unless it is contextually clear either one of the elements or more than one of the elements is described.

The term “about” as used herein refers to a value within 10% of the underlying parameter (i.e., plus or minus 5%), and use of the term “about” at the beginning of a string of values modifies each of the values (i.e., “about 1, 2 and 3” refers to about 1, about 2 and about 3). Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

As used herein, the term “substantially” refers to approximately the same shape as stated.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments.

Trademarks: the product names used in this document are for identification purposes only; and are the property of their respective owners.

### LIST OF REFERENCES CITED

1. Oltulu, P., Ince, B., Kokbudak, N., Findik, S. and Kilinc, F. (2018). Measurement of epidermis, dermis and total skin thickness from six different body regions with a new ethical histometric technique. Turkish Journal of Plastic Surgery, 26, 56-61.
2. Montecucco C., and Schiavo, G. (1995). Structure and function of tetanus and botulinum neurotoxins. Q Rev Biophys., 28, 423-472.
3. Li L, and Singh B. R. (2000). Spectroscopic analysis of pH-induced changes in the molecular features of type A botulinum neurotoxin light chain. Biochemistry, 39, 6466-6474.
4. Segelke B., Knapp M., Kadkhodayan S., Balhorn R., and Rupp B. (2004).Crystal structure of Clostridium botulinum neuro toxin protease in a product-bound state: Evidence for noncanonical zinc protease activity. PNAS, 101, 6888-6893.
5. Silvaggi N., Boldt, G. E., Hixon M. S., Kennedy J. P., Tzpori S., Janda K. D., and Allen K. N. (2007) Structures of Clostridium botulinum Neurotoxin Serotype A Light Chain Complexed with Small-Molecule Inhibitors Highlight Active-Site Flexibility. Chemistry and Biology, 14, 533-542.
6. Li L., and Singh B. R. (1999). High-level expression, purification, and characterization of recombinant type A botulinum neurotoxin light chain. Protein Expr Purif. 17, 339-44
7. Kukreja R. V., and Singh B. R. (2007). Comparative role of neurotoxin-associated proteins in the structural stability and endopeptidase activity of botulinum neurotoxin complex types A and E. Biochemistry, 46,14316-24.
8. Inoue K, Fujinaga Y, Watanabe T etal. Molecular composition of Clostridium botulinum type A progenitor toxins. Infect. Immun. 1996, 64, 1589-1594.
9. Singh B R, Chang T W, Kukreja R and Cai S. The Botulinum Neurotoxin Complex and the Role of Ancillary Proteins. In: Molecular Aspects of Botulinum Neurotoxin (Foster, Keith. A., Ed.), Springer, New York. Pp. 2014, 69-102.
10. Singh B R, Foley J, and Lafontaine C. Physico-chemical characterization of the botulinum neurotoxin binding protein from type E botulinum purified from Clostridium botulinum. J. Protein Chem 1995, 14, 7-18.
11. East A K, and Collins M D. Conserved structure of genes encoding components of the botulinum neurotoxin complex M and the sequence of the gene encoding for the nontoxic component in nonproteolytic Clostridium botulinum type F. Curr Microbiol 1994, 29, 69-77.
12. Carli L, Montecucco C, and Rossetto O. Assay of diffusion of different botulinumneurotoxin type A formulations injected in the mouse leg. Muscle Nerve 2009. 40, 374-380.
13. Stone H F, Zhu Z, Thach T Q, and Ruegg C L. Characterization of diffusion and duration of action of a new botulinum toxin type A formulation. Toxicon 2011, 58,159-167.
14. Panicker J N, and Muthane J B. Botulinum toxins: Pharmacology and its current therapeutic evidence for use. Neurol India 2003, 51, 455-460.
15. Kim E J, Ramirez A L, Reeck J B, and Maas C S. The role of botulinum toxin type B (Myobloc) in the treatment of hyperkinetic facial lines. Plast Reconstr Surg. 2003,112, 88S-93S; discussion 94S-97S.
16. G. L. Amidon, H. Lennernas, V. P. Shah, J. R. Crison. A theoretical basis for a biopharmaceutic drug classification: the correlation in vitro drug product dissolution and in vivo bioavailability. Pharm Res, 12 (1995), 413-420.
17. V. Jannin, J. Musakhanian, D. Marchaud. Approaches for the development of solid and semi-solid lipid-based formulations. Adv Drug Deliv Rev, 60 (2008), 734-746.
18. Habermann, E., Müller, H., & Hudel, M. (1988). Tetanus toxin and botulinum A and C neurotoxins inhibit noradrenaline release from cultured mouse brain. Journal of neurochemistry, 51(2), 522-527.
19. Pearce, L. B., First, E. R., Maccallum, R. D., & Gupta, A. (1997). Pharmacologic characterization of botulinum toxin for basic science and medicine. Toxicon, 35(9), 1373-1412.
20. Bigalke H., Dreyer F., and Bergey G. K. (1985) Botulinum A neu-rotoxin inhibits non-cholinergic synaptic transmission in mouse spinal cord neurons in culture. Brain Res. 360, 3 18-324.
21. Bigalke H., Miiller H., and Dreyer F. (1986) Botulinum A, unlike tetanus toxin, acts via a neuraminidase sensitive structure. Toxicon 24, 1065-1074.
22. Jankovic, J. (1994). Botulinum toxin in the treatment of dystonic tics. Movement disorders: official journal of the Movement Disorder Society, 9(3), 347-349.
23. Kumar, R., Chang, T. W., and Singh, B. R. (2015). Evolutionary traits of toxins. P. Gopalakrishnakone et al. (eds.), Biological Toxins and Bioterrorism, Toxinology, DOI 10.1007/978-94-007-5869-8_29, 527-557.

What is claimed is:

1. A pharmaceutical composition comprising one or more proteins of Clostridium botulinum in a nano-emulsion for topical delivery.

2. The pharmaceutical composition of claim 1, wherein the protein comprises one or more from botulinum toxin A (BoNT/A), BoNT/B, BoNT/C, BoNT/D, BoNT/E. BoNT/F, or BoNT/G, or their different forms.

3. The pharmaceutical composition of claim 1, wherein the proteins are botulinum toxins or its complex or its other forms.

4. The pharmaceutical composition of claim 1, wherein the pharmaceutical composition further comprises of other proteins of Clostridium botulinum.

5. The pharmaceutical composition of claim 1, wherein the pharmaceutical composition comprises of any other therapeutic proteins including biomolecules, vaccines, antibodies and protein hormones.

6. The pharmaceutical composition of claim 1, wherein the nano-emuslisons are encapsulated microspheres and nanospheres containing propylene glycol (0.5 to 5% w/v), phenoxyethanol (0.1 to 1% w/v), sodium hyaluronate (0.1 to 1% w/v), caprylic/capric triglyceride (0.5 to 5% w/v), hydrogenated castor oil (0 to 10% w/v), span-80 (0.1 to 1% w/v) and saponin (1 to 10% w/v) with water.

7. The pharmaceutical composition of claim 1, wherein the pharmceutical composition further comprises surface active agents, chelating agents, salicylates, anti-inflammatory agenets, antibacterial agents, antifungal agents, antiviral agents or phenothiazines and bioactive peptides.

8. The pharmaceutical composition of claim 7, wherein the bioactive agent comprises one or more of a: pentapeptide KTTKS, tetrapeptide GQPR, hexapeptide argireline, tripeptide GHK, Snap-8 octapeptide and oligopeptides.

9. The pharmaceutical composition of claim 1, further comprising one or more of: oil triglyceride, ethyl icosapentate, castor oil, tocopherol nicotinate, teprenone, indomethacin franesil, soy-bean oil, tea oil, sunflower seed oil, vegetable oil, fish oil, sesame oil, soybean oil, tea oil, sunflower seed oil, vegetable oil, fish oil, sesame oil, soy-bean oil, tea oil, sunflower seed oil, vegetable oil, fish oil, sesame oil, soy-bean oil, tea oil, sunflower seed oil, vegetable oil, fish oil, sesame oil, Labrafac Lipophile WL 1349 oil, and dronabinol).

10. The pharmaceutical composition of claim 1, further comprising one or more excipients comprising: oil solution, mixed glycerides, water-soluble co-solvents and surfactants, and self-made creams.

11. The pharmaceutical composition of claim 10, wherein the surfactants comprise one or more of: Permulen TR1, Permulen TR2, RH-40, Tween-80, and Tween-60.

12. The pharmaceutical composition of claim 1, wherein the pharmaceutical composition further comprises humectants and emolliants.

13. The pharmaceutical composition of claim 12, wherein the humectants comprise propylene glycol or lecithin; and the emolliants comprise zinc oxide or dimethicone.

14. The pharmaceutical composition of claim 1, wherein the pharmaceutical composition further comprises a stabilizer.

15. The pharmaceutical composition of claim 14, wherein the stabilizer comprises human serum albumin or IgG.

16. The pharmaceutical composition of claim 1, wherein the pharmaceutical composition is a lyophilized powder, lotion, serum or gel form.

17. The pharmaceutical composition of claim 1, wherein the proteins within the pharmaceutical composition are emulsified and mixed with one or more of: retinoids, alpha hydroxyl acids, hyaluronic acid and/or its sodium salt, resveratrol, stem cells, EGFs (epidermal growth factors), KGFs (keratinocyte growth factors), FGFs (fibroblast growth factors, HGH (human growth hormones), niacinamide, aloe vera, allantoin and therapeutic agents.

18. The pharmaceutical composition of claim 1, wherein the pharmaceutical composition is stabilized at a pH in between 5.5 and 8.0.

19. The pharmaceutical composition of claim 1, further comprising components for administering topically.

20. The pharmaceutical composition of claim 1, wherein the pharmaceutical composition is able to effectively treat local and systemic conditions comprising: neuromuscular, gastrointestinal, diabetic, cardiovascular, reproductive issues, skin problems; and to work as a local aesthetic.

21. A method of treatment comprising,
a. topically administering a nano-emulsion pharmaceutical composition to a subject, comprising one or more proteins derived from Clostridium botulinum;
b. wherein the one or more proteins comprise one or more from botulinum toxin A (BoNT/A), BoNT/B, BoNT/C, BoNT/D, BoNT/E. BoNT/F, or BoNT/G, or their different forms, or any combination thereof; and
c. wherein the pharmaceutical composition effectively treats local and systemic conditions comprising: neuromuscular, gastrointestinal, diabetic, cardiovascular, reproductive issues, skin problems; and/or to work as a local aesthetic.

22. A method of preparing an encapsulation of a toxin or complex, comprising the steps:
(1) preparing a solution A by mixing sodium hyaluronate, propylene glycol, Tween-80 and saponin in water by continuous stirring;
(2) preparing a solution B by mixing phenoxy ethanol and, carylic acid or capric triglyceride, together;
(3) mixing solution B into solution A by continuous stirring for 15 minutes to form solution C;
(4) mixing Botulinum toxin and 10 mM Sodium Phosphate buffer to a pH of 7.1, and adding to solution C; and
(5) stirring solution C at room temperature continuously for 15-20 minutes.

23. The method of preparing of claim 22, further comprising adding hydrogenated castor oil to solution B.
